(19) United States
(12) Patent Application Publication
(10) Pub. No.: US 2006/0094765 A1
(43) Pub. Date: May 4, 2006

(54) Use of a sri and vitamin b6 for the treatment of neurological and mental disorders

(75) Inventors: Herman Jan Tijmen Coelingh Bennink, Maarn (NL); Rene Frank Van Der Linden, Maarn (NL)

Correspondence Address: THE WEBB LAW FIRM, P.C., 700 KOPPERS BUILDING, 436 SEVENTH AVENUE, PITTSBURGH, PA, 15219, US

(73) Assignee: Pantarhei Bioscience B.V., Zeist (NL)

(21) Appl. No.: 10/526,849

(22) PCT Filed: Sep. 5, 2003
(86) PCT No.: PCT/NL03/00622

§ 371 Date: Sep. 23, 2005

(30) Foreign Application Priority Data
Sep. 5, 2002 (NL) 02078645.5

(51) Int. Cl. A61K 31/4415

(52) U.S. Cl. 514/350

(57) ABSTRACT

The invention is concerned with a method of treating neurological and/or mental disorders that are associated with, or pathogenetically related to deficient serotonin neurotransmission. The invention relates to a method of treating depressive disorders, anxiety disorders, post-traumatic stress syndrome or premenstrual syndrome, said method comprising the administration of serotonin re-uptake inhibitor (SRI) in a daily amount of at least 0.4 mg, wherein a vitamin B6 component is co-administered in a daily amount of between 0.01 and 10 mmoles to improve the responder rate or to advance the onset of action of the treatment with SRI, and wherein the method does not include the application to the brain of an AC pulsed magnetic field of at least 7.5 picotesia flux density for at least 15 minutes. The invention also encompasses pharmaceutical formulations containing SRI and vitamin B6 component and comprising a dosage unit that provides a sustained release of the vitamin B6 component.

### FIELD OF THE INVENTION

The present invention is concerned with a method of treating neurological and/or mental disorders that are associated with, or pathogenetically related to deficient serotonin neurotransmission. More particularly the invention relates to a method of increasing cerebral intercellular serotonin levels and/or of preventing decreases in such serotonin levels by a method that comprises administering a serotonin re-uptake inhibitor.

### BACKGROUND OF THE INVENTION

Serotonin (5-hydroxytryptamine) is a neurotransmitter that is found in relatively high concentrations in the lateral gray horns of the spinal cord and in a number of areas in the brain. A system of serotonin-containing neurons that have their cell bodies in the raphe nuclei of the brain stems project to portions of the hypothalamus, the limbic system, the neocortex and the spinal cord. It has been demonstrated that serotonin interacts with a great number of receptors in the brain and controls or affects processes which regulate many bodily organs and functions.

Serotonin is an important chemical messenger participating in the transmission of nerve impulses in the brain. These messengers are released at specific sites on pre-synaptic cells and received, to complete transmission of the impulse, at specific sites on post-synaptic cells. Their effect is then terminated by metabolism or by uptake into the pre-synaptic cells. In addition, some of the released serotonin is inactivated by monoamine oxidase to form 5-hydroxyindoleacetic acid. This is the principal urinary metabolite of serotonin.

Serotonin is formed in the body by hydroxylation and decarboxylation of the essential amino acid L-tryptophan. In the biosynthesis of serotonin from L-tryptophan, L-tryptophan is hydroxylated in the presence of the enzyme tryptophan hydroxylase to form the intermediate product L-5-hydroxytryptophan. This intermediate product is decarboxylated in the presence of the enzyme 5-hydroxytryptophan decarboxylase to from serotonin.

Serotonin deficiencies in the brain have been associated with a number of mainly psychological disorders such as (endogenous) depression, insomnia, excessive appetite and lowered pain threshold.

An important discovery in medicinal chemistry of the past decades are the so called serotonin re-uptake inhibitors (SRI's), which are particularly effective in the treatment of depression. SRI's increase the availability of serotonin in the synapse by reducing the uptake of serotonin by the serotonin uptake carrier (transporter protein). Dysfunction of the serotonin neurons resulting from excessive uptake results in depression, as well as other pathologies of the central nervous system.

Among the commercially available SRI's are fluoxetine, nefazodone, sertraline, venlafaxine, citalopram, fluvoxamine, paroxetine and trazodone. While the primary activity of these drugs is the inhibition of the re-uptake of serotonin, the cascade of monoamine processes in the brain connects serotonin with both norepinephrine and dopamine. Thus, the increase of availability of serotonin may result in increased availability of norepinephrine and dopamine as well.

Since the commercial introduction of the first SRI's in the early eighties these pharamaceutical components have found widespread application, particularly as anti-depressants. Despite this success there are a few serious drawbacks to the use of SRI's that have not yet been resolved. One such drawback relates to the fact that after administration of SRI has commenced, it usually takes a few weeks before its therapeutic impact (action) becomes apparent. Consequently, these drugs are less suited for treating individuals who suffer from non-chronic afflictions or for treating individuals who need urgent relief (e.g. suicidal patients).

Another drawback of known SRI's is related to the relatively low responder rate that has been observed for these drugs. In general it is found that only about 60-70% of the patients treated with SRI respond well to such treatment. Given that it takes a few weeks before it can be established whether or not a patient responds well to treatment with SRI, it will be clear that better responder rates are highly desirable.

In view of the aforementioned drawbacks there is a need for a method of treatment that will deliver the functionality of SRI's more quickly and/or with a higher responder rate. It is an objective of the present invention to make available a method that meets these requirements.

### SUMMARY OF THE INVENTION

The inventors have surprisingly found that the aforementioned objective can be realised by a method of treatment that comprises the administration of an effective amount of a combination of SRI and a vitamin B6 component. The co-administration of said vitamin B6 component(s) was unexpectedly found to advance the onset of action of the SRI. Also, the co-administration of the vitamin B6 component improves the responder rate of a given SRI.

The present method can advantageously be used for the treatment of any disorders or diseases for which SRI's have been applied successfully. Such disorders and diseases include depressive disorders, anxiety disorders, post-traumatic stress syndrome and premenstrual syndrome.

Although the combined use of SRI's and vitamin B6 has been described in the prior art, it has not been recognised before that vitamin B6 has the capability of advancing the onset of the action of SRI's or of increasing the responder rate of these drugs.

U.S. Pat. No. 4,596,807 (Crosby) describes a method for controlling pain, depression and sedation, which method comprises administering a composition comprising a serotonin precursor, such as L-tryptophan or L-5-hydroxytryptophan in an amount effective to increase the brain serotonin to a supra normal level, in combination with a SRI in an amount effective to inhibit the re-uptake of serotonin. The composition may additionally comprise vitamin B6 (pyridoxine) and vitamin C (ascorbic acid) so as to aid biosynthesis of serotonin in case of patients suffering from vitamin depletion. In accordance with this patent administration of the composition is desirably effected in 1-4 portions daily, delivering 100-800 mg per day of SRI and 40-400 mg per day of the pyridoxine.

U.S. Pat. No. 5,885,976 (Sandyk) is concerned with methods for the treatment of neurological and mental disorders related to deficient serotonin neurotransmission and impaired pineal melatonin functions. The method described comprises administering an effective amount of a composition which increases serotonin transmission followed by the application to the brain of a sufficient amount of an AC pulsed magnetic field to treat the disorder. It is mentioned that the composition may contain a stimulant of serotonin synthesis which is vitamin B1, vitamin B3, vitamin B6, biotin, S-adenosyl methionine, vitamin D, folic acid, ascorbic acid, magnesium, coenzyme Q10, piracetam, or mixtures of two or more thereof. Furthermore it is stated that the composition can include a SRI which is sertraline, nefazodone, trazodone, fluoxetine or a mixture thereof. The ranges of daily dosage levels specifically mentioned for a number of SRI's in this patent broadly cover the range of 25-600 mg.

U.S. 2002/0072537 describes a method to facilitate weight loss for a patient comprising administration of citalopram and phentermine. The US-application mentions that on start-up, to minimise the possibility of medication ineffectiveness the patient should consume 50 to 200 mg of 5-Hydroxytryptophan a day, Vitamin B6 in the dosing range of 2 to 150 mg per day, Vitamin C in the dosing range of 50 to 2000 mg per day andd optionally Tyrosine in the dosing range of 50 to 4000 mg per day as well as Calcium in the dosing range of 50-2000 mg per day and Lysine in the dosing range of 50 to 2000 mg per day.

These prior art publications do not contain any suggestions that co-administration of vitamin B6 may advance the onset of action of SRI's or improve the responder rate of treatment of depression or anxiety disorders with SRI's.

### DETAILED DESCRIPTION OF THE INVENTION

Accordingly, one embodiment of the present invention relates to a method of treating depressive disorders, anxiety disorders, post-traumatic stress syndrome or premenstrual syndrome, by increasing cerebral intercellular serotonin levels and/or of preventing decreases in such serotonin levels, said method comprising the administration of serotonin re-uptake inhibitor in a daily amount of at least 0.4 mg, preferably of between 0.4 and 80 mg, wherein vitamin B6 component is co-administered in a daily amount of between 0.01 and 10 mmoles to improve the responder rate or to advance the onset of action of the treatment with SRI, and wherein the method does not include the application to the brain of an AC pulsed magnetic field of at least 7.5 picotesia flux density for at least 15 minutes. More preferably the method does not include the application of an AC pulsed magnetic field of at least 7.5 picotesia Most preferably the method does not include the application of an AC pulsed magnetic field at all.

The co-administration of vitamin B6 component according to the present invention is particularly effective in advancing the onset of action of the SRI treatment. This is particularly relevant in the treatment of depression and anxiety disorders as patients suffering from such disorders are seeking instant solace. The benefits of the present invention are particularly appreciated when the method is used to treat patients suffering from non-chronic depression or anxiety disorders.

The term “serotonin re-uptake inhibitor”, also sometimes referred to as “serotonin selective re-uptake inhibitor (or SSRI)” encompasses those re-uptake inhibitors that are capable of significantly inhibiting re-uptake of serotonin by blocking its transporter protein, by inhibiting monoamine oxidase and/or by (selectively) blocking cerebral serotonin receptors. Preferably the SRI used in the present method is capable of blocking the serotonin transporter protein.

The term “on a daily basis”, when used in connection with a mentioned dosage amount, should not be interpreted restrictedly. For instance, the above mentioned requirement that the administration of the present formulation is to provide, on a daily basis, at least 0.4 mg SRI, encompasses a protocol wherein SRI is administered once a week, provided the weekly dosage is at least 2.8 mg, i.e. such that the average daily dose is at least 0.4 mg SRI.

The term “vitamin B6 component” as used throughout this document encompasses any components which in vivo, particularly once these components or their metabolites have entered the bloodstream, are converted into pyridoxal or a pyridoxal salt. Particularly useful are vitamin B6 components that in vivo are converted for at least 10 mol % into pyridoxal or a pyridoxal salt within 24 hours after administration.

Inside living human and animal cells, pyridoxal phosphate and pyridoxamine phosphate are the biologically active forms of vitamin B6, acting as a co-enzyme in more than 100 biological reactions. In the form of pyridoxal 5′-phosphate and pyridoxamine 5′-phosphate, vitamin B6 acts as the coenzyme of a series of enzymes, which catalyse transamination, decarboxylation, deamination, desulphydration and the cleavage or synthesis of amino acids. The aminotransferases represent an important link between amino acid, carbohydrate and fatty acid metabolism and the energy-producing citric acid cycle. The decaboxylases convert amino acids to the corresponding biogenic amines, such as histamine, hydroxytyramine, serotonin, γ-aminobutyric acid, ethanolamine and taurine, some of which are substances of high physiological activity [regulation of the blood vessel diameter, neurohormonal actions, essential components of phospholipids and bile acids]. In accord with the general importance these enzymatic reactions take place in virtually all organs most intensively in the liver, heart and brain. (Lit. Vitamin Compendium; Roche, Vitamins and Chemicals Department. F. Hoffmann-La Roche and Co. Ltd, Basle, Switzerland).

As described herein before SRI and the vitamin B6 component may be co-administered in a sequential as well as in a coextensive fashion. Hence the term “pharmaceutical formulation” as used throughout this document should be construed to encompass pharmaceutical kits which comprise separate dosage units for the SRI component and the vitamin B6 component Preferably, however, the SRI component and vitamin B6 component are combined into a single dosage unit.

The SRI and vitamin B6 component may suitably be administered enterally (e.g. orally or rectally), parenterally (e.g. intravenous or intramuscular) or topically. Preferably, the present method employs enteral administration, most preferably oral administration. The present method may suitably be used to treat a wide variety of mammals. The method, however, is particularly suitable for treating humans.

The present method is particularly useful for treating depression or anxiety disorders (e.g. generalised anxiety disorder (GAD), panic disorder and social anxiety disorder (social phobia)). Most preferably the present method is used for the treatment of depression. The present method does not encompass the treatment of hot flushes in e.g. hypo-estrogenic females and androgen-deprived males.

Depression is often associated with other diseases and conditions, or caused by such other conditions. Depression may also be associated with abuse of any substance, or may be associated with behavioural problems resulting from or occurring in combination with head injuries, mental retardation or stroke. Depression in all its variations is a target of treatment for formulations according to the present invention.

According to another preferred embodiment of the method of treatment according to the invention said method comprises at least once daily administration of the pharmaceutical formulation. Since SRI's are metabolised relatively slowly, it is possible to apply longer intervals between doses. However, longer intervals will usually lead to an increase in amplitude of the observed fluctuations of SRI blood serum levels. Since a minimum blood serum level needs to be maintained to achieve the desired results, the maximally occurring blood serum level of SRI will inevitably increase with incrementing dosage intervals. This is not in line with the aim of the present invention to not employ unnecessarily high SRI blood serum levels.

The present method of increasing cerebral serotonin levels offers the advantage that it employs relatively low levels of SRI. Thus in comparison to ordinary methods, employing higher dosage of SRI, the present method exhibits a decreased incidence of side-effects. This may be explained from the fact that the vitamin B6 component, in the proposed dose ranges, has no side-effects. In a preferred embodiment of the invention the method provides on a daily basis a minimum amount of 0.6 mg SRI. The preferred daily maximum amount is 70 mg SRI. More preferably the daily maximum amount is 60 mg SRI, even more preferably it is 50 mg. Most preferably the maximum daily amount of SRI provided by the method is 40 mg. The daily minimum amount provided by the present method preferably is 1 mg, more preferably 2 mg and most preferably 3 mg.

According to another preferred embodiment the present method provides on a daily basis a minimum amount of 3.0 mg SRI, calculated as trazodone equivalent. More preferably the daily minimum amount is 10.0 mg trazodone equivalent, most preferably 15.0 mg trazodone equivalent. The preferred daily maximum amount is equivalent to less than 90 mg trazodone, more preferably less than 80 mg trazodone. Most preferably the maximum daily amount of SRI provided by the method is equivalent to less than 75 mg trazodone.

In order to facilitate the translation of given amounts of trazodone into the equivalent amounts of another SRI and vice versa, the following table provides the conversion factors that are to be used:

| | Trazodone conversion factor |
|---|---|
| citalopram | 0.15 |
| fluoxetine | 0.15 |
| fluvoxamine | 0.7 |
| paroxetine | 0.15 |
| sertraline | 0.3 |
| venlafaxine | 0.5 |
| trazodone | 1.0 |
| mirtazapine | 0.2 |
| nefazodone | 1.3 |
| escitalopram | 0.04 |
| duloxetine | 0.15 |

With the help of the above table it can be calculated that 100 mg trazodone is equivalent to 30 mg sertraline or 15 mg fluoxetine.

The present formulations preferably do not contain a narcotic, such as those mentioned in U.S. Pat. No. 4,596,807 (Crosby). More particularly the present method does not encompass the administration of a narcotic selected from the group consisting of codeine, oxycodone, propoxyphene, pentazocine, morphine, meperidine, levorphanol, menthadone and mixtures thereof, in an amount effective to produce analgesia.

The present method of treatment preferably comprises administration of SRI in a daily amount of between 0.01 and 1 mg SRI per kg bodyweight and vitamin B6 component in a daily amount of between 0.001 and 0.2 mmoles per kg bodyweight. More preferably the SRI is administered in a daily amount of between 0.02 and 0.8 mg SRI/kg and the vitamin B6 component in a daily amount of between 0.004 and 0.1 mmoles/kg. Most preferably the SRI is administered in a daily amount of between 0.03 and 0.7 mg SRI/kg.

Analogues of vitamin B6 which can suitably be used in accordance with the present invention are those selected from the group consisting of pyridoxal, pyridoxamine, acetals of pyridoxal, condensation products arising from the reaction of the aldehyde group of pyridoxal with an amine, and addition salts of any of the foregoing members of the group with pharmaceutically acceptable salts. The term “pharmaceutically acceptable salts” includes salts with pharmaceutically acceptable acids of bases, e.g. acids such as sulphuric, hydrochloric, nitric, phosphoric acid, etc. or bases such as alkali or alkaline earth metal hydroxides, ammonium hydroxides, alkyl ammonium hydroxides etc.

In order to obtain a significant beneficial effect from the co-administration of SRI and vitamin B6 component, it is advisable to apply the vitamin B6 component and SRI in a dosage ratio of at least 0.001 mmole/mg, preferably of at least 0.01 mmole/mg, more preferably of at least 0.03 mmole/mg and most preferably of at least 0.05 mmole/mg. Generally the aforementioned ratio will not exceed 2 mmole/mg, preferably it will not exceed 1 mmole/mg and more preferably it will not exceed 0.5 mmole/mg.

In a preferred embodiment of the present invention the SRI's employed are selected from the group consisting of citalopram, escitalopram, fluoxetine, norfluoxetine, fluvoxamine, paroxetine, sertraline, venlafaxine, zimelidine, femoxetine, trazodone, nefazodone, mirtazapine, duloxetine, pharmaceutically acceptable salts of these inhibitors and mixtures thereof. More preferably, the SRI's are selected from the group consisting of citalopram, fluoxetine, norfluoxetine, fluvoxamine, paroxetine, sertraline, venlafaxine, zimelidine, femoxetine, trazodone, nefazodone, mirtazapine, pharmaceutically acceptable salts of these inhibitors and mixtures thereof. Most preferably the SRI's are selected from the group consisting of citalopram, fluoxetine, fluvoxamine, paroxetine, sertraline, venlafaxine, zimelidine, trazodone, nefazodone, mirtazapine, pharmaceutically acceptable salts of these inhibitors and mixtures thereof. It is noted that the SRI's used in present formulations may be present in the form of a racemic mixture as well as in the form of a stereo-enantiomer.

In another preferred embodiment of the present invention the pharmaceutical formulation additionally comprises at least 300 mg, more preferably from 500 to 10,000 mg of a serotonin precursor selected from the group consisting of L-tryptophan, 5-hydroxytryptophan, precursors of these tryptophan substances and mixtures thereof. The inclusion of the latter precursors may assist in alleviating serotonin deficiency since serotonin is biosynthesised from tryptophane through the following metabolic chain: tryptophan>5-hydroxytryptophan>5-hydroxytryptamine>serotonin.

Tryptophan is usually not transported in the blood in a free state, but rather bound to or complexed with blood serum albumin. Tryptophan is the only circulating amino acid that is significantly bound to human blood serum albumin. It has been shown that salicylates displace tryptophan from its protein binding site on albumin in blood plasma thereby raising the free, circulating tryptophan concentration in blood. This free or unbound tryptophan is more easily converted to serotonin than the bound form. Hence the present pharmaceutical formulation may advantageously contain as an additional component a salicylate. Preferably the formulation comprises at least 0.05 mmoles of such a salicylate, more preferably from 0.1 to 1.5 mmoles of the salicylate. Here the term salicylate includes both the acid and the salt. The salicylate is preferably selected from the group consisting of sodium salicylate, choline salicylate, magnesium salicylate and mixtures thereof.

Yet another component which may additionally be included in the present formulation is a component that acts as a stimulant of the serotonin receptor, such as buspirone. Hence in another preferred embodiment the formulation comprises at least 10 μmoles, preferably from 20 to 300 μmoles of buspirone.

The pharmaceutical formulations according to the invention can be solid or semi-solid dosage forms such as tablets, capsules, cachets, pellets, pills, suppositories, powders and granules, as well as fluid dosage forms such as solutions, emulsions, suspensions, ointments, pastes, creams, gels, jellies and foams. In addition to the pharmacologically active components, the formulation according to the invention contains pharmaceutically acceptable excipient, usually in an amount of between 50 and 99.9 wt. %.

Tablets and equivalent solid and semi-solid dosage forms can suitably contain excipients such binders (e.g. hydroxypropylmethyl cellulose, polyvinyl-pyrrolidine, other cellulosic materials and starch), diluents (e.g. lactose and other sugars, starch, dicalcium phosphate and cellulosic materials), disintegrating agents (e.g. starch polymers and cellulosic materials) and lubricating agents (e.g., stearates and talc).

Transdermal delivery systems include patches, gels, tapes and creams, and can contain excipients such as solubilisers, permeation enhancers (e.g. fatty acids, fatty acid esters, fatty alcohols and amino acids), hydrophilic polymers (e.g. polycarbophil and polyvinyl pyrollidine and adhesives and tackifiers (e.g. polyisobutylenes, silicone-based adhesives, acrylates andpolybutene).

Transmucosal delivery systems include patches, tablets, suppositories, pessaries, gels, and creams, and can contain excipients such as solubilizers and enhancers (e.g. propylene glycol, bile salts and amino acids), and other vehicles (e.g. polyethylene glycol, fatty acid esters and derivatives, and hydrophilic polymers such as hydroxypropylmethyl cellulose and hyaluronic acid).

Injectable delivery systems include solutions, suspensions, gels, microspheres and polymeric injectables, and can comprise excipients such as solubility-altering agents (e.g. ethanol, propylene glycol and sucrose) and polymers (e.g. polycaprylactones, and PLGA's). Implantable systems include rods and discs, and can contain excipients such as PLGA and polycapryl lactone.

Other delivery systems that can be used for administering the pharamaceutical composition of the invention include intranasal delivery systems such as sprays and powders, and sublingual delivery systems.

The inventors have observed that in order to obtain the full benefit from the co-administration of the vitamin B6 component it is advantageous to administer said component at relatively short time intervals, e.g. every 4-6 hours. It is believed that, following absorption, vitamin B6 is quickly converted into metabolites that have less of a synergistic interaction with the SRI. Consequently, it is advantageous to administer the vitamin B6 component by means of a sustained release formulation, so as to avoid the burden of having to administer said component 4-6 times per day. As a result of the controlled release from a sustained release formulation (e.g. an oral dosage form, a suppository or a transdermal patch) the blood serum level of the vitamin B6 component can be maintained at a stable level which coincided with the level that produces the optimum synergistic interaction between the SRI and the vitamin B6 component.

The present invention encompasses an embodiment wherein the vitamin 136 component is separately administered from the SRI in the form of a slow release formulation. Preferably, however, both active agents are administered in the form of a slow release formulation. Most preferably both agents are administered through a single slow release formulation, in particular a single solid dosage form.

Generally the present sustained release formulation should release the vitamin B6 component at a rate of at least 0.001 mmole/hour during the first 4 hours after administration. Preferably the release rate during said period is at least 0.005 mmole/hour, more preferably at least 0.01 mmole/hour.

In a particularly preferred embodiment the present method comprises the administration of a sustained release formulation at intervals of at least one day. More preferably, said intervals are within the range of 1 day and 1 week. Most preferably the sustained release formulation is administered once daily.

Another embodiment of the present invention relates to a pharmaceutical formulation for oral, rectal, buccal or transdermal administration, comprising a solid dosage form that contains as active agents at least 0.4 mg of serotonin re-uptake inhibitor and at least 0.01 mmole of vitamin B6 component as well as a pharmaceutically acceptable carrier, said dosage unit providing a sustained release of the vitamin B6 component, e.g. by means of a membrane coating material, that provides for sustained release of these active agents from the dosage form. The term “solid dosage unit” also encompasses dosage units that e.g. comprise a liquid or a paste which is encased in a capsule.

The pharmaceutical formulation according to the present invention may suitably take the form of an oral dosage unit, a buccal dosage unit, a suppository, a transdermal patch or a depot injection. Preferably said formulation comprises an oral dosage unit or a suppository. Most preferably the present formulation comprises an oral dosage unit.

In order to ensure the sustained release of the active agents, the present formulation may advantageously comprise a membrane coating material that separates the active agents from the exterior. In case the formulation is administered orally, buccally or rectally, the active agents will gradually diffuse through said membrane coating and/or said coating will gradually degrade allowing release of the active agents from the interior of the formulation.

In a particularly preferred embodiment, the solid dosage form is effective to provide a sustained release profile wherein less than 50 wt. % of the vitamin B6 component is released from the dosage form within the first 4 hours after administration and more than 80 wt. % of the vitamin B6 component is released from the dosage form within 24 hours.

Yet another aspect of the present invention relates to a pharmaceutical formulation containing at least 0.4 mg of SRI, at least 0.01 mmole of vitamin B6 component and a pharmaceutically acceptable carrier, said vitamin B6 component being contained in a dosage unit that provides a sustained release of the vitamin B6 component.

In a particularly preferred embodiment, vitamin B6 component and SRI are present in the present pharmaceutical formulations in a ratio of between 0.01 and 1 mmole/mg, preferably in a ratio of between 0.05 and 0.5 mmole/mg. The application of a relatively high ratio of vitamin B6 component to SRI ensures that optimum responder rates are achieved at a given SRI dosage.

The invention is further illustrated by means of the following examples.

### EXAMPLE

### Example I

Chronic mild stress (CMS), a well-validated preclinical model of depression (Willner, 1997, Validity, reliability and utility of the chronic mild stress model of depression: a 10-year review and evaluation. Psychopharmacology, 134, 319-329, Nestler et al., 2002, Preclinical models: status of basic research in depression, Soc. Biol. Psych., 52, 503-528), was used to study the effects of combined therapy of SRI with pyridoxine on responder rate and the time to achieve a full drug response (‘onset of action’) as compared to treatment with SRI alone. Citalopram, as an example of a SRI, was combined with either pyridoxine or vehicle and administered to chronically, mildly stressed rats during a 5-week treatment schedule. Consumption of sucrose solution after fasting, which a commonly accepted measure of anhedonia, i.e. inability to experience pleasure, was chosen for readout, since this is a core symptom of human depressive disorders.

Male Wistar rats were brought into the laboratory two months before the start of the experiment for acclimatization and training (see below). During this period, the animals were singly housed and were maintained on a 12-h light/dark cycle (lights on at 08.00) at a constant temperature of 22±2° C. and humidity (55±5%). Food pellets (containing an optimal level of 1 mg/kg pyridoxine) and water were freely available, with the exception of the deprivation procedure as described below.

During a period of 5 weeks, rats were trained to consume a 1% sucrose solution. Training consisted of ten 1-h baseline tests (twice weekly) in which sucrose was presented, in the home cage, following 14 h of food and water deprivation. Sucrose intake was measured by weighing pre-weighed bottles containing the sucrose solution, at the end of the test. Subsequently, sucrose consumption was monitored, under similar conditions, at weekly intervals throughout the whole experiment.

On the basis of sucrose intake in the final baseline test, animals were divided into two matched groups. One group of animals was subjected to the chronic stress procedure for a period of seven consecutive weeks according to the procedure described by Papp and coworkers (Papp et al., 2003, Effect of agomelatine in the chronic mild stress model of depression in the rat. Neuropsychopharmacology, 28, 694-703; and references herein). Each week of the stress regime consisted of: two periods of food or water deprivation, two periods of 45 degree cage tilt, two periods of intermittent illumination (lights on and off every 2 h), two periods of soiled cage (250 ml water in sawdust bedding), two periods of paired housing, two periods of low intensity stroboscopic illumination (150 flashes/min), and two periods of no stress in random order. All stressors were 10-14 h of duration and were applied individually and continuously, day and night. Control animals were housed in separate rooms and had no contact with the stressed animals. They were deprived of food and water for 14 h preceding each sucrose test, but otherwise food and water were freely available in the home cage.

On the basis of sucrose intake scores following the first 2 weeks of stress, both stressed and control animals were redivided into matched subgroups (n=8 per group). During the subsequent five weeks the animals received daily drug treatment by intraperitoneal injections of either vehicle (distilled water, 1 ml/kg), citalopram (2.5 or 5.0 mg/kg), or citalopram (2.5 or 5.0 mg/kg) combined with pyridoxine (10 mg/kg). All drugs were administered in a volume of 1 ml/kg body weight at approximately 10.00 am. The weekly sucrose tests were carried out 24 h following the last drug injection. Except for control animals, stress was continued throughout the period of drug treatment. After five weeks treatment the study was terminated.

Mean values in sucrose consumption during stress and subsequently the 5 weeks of vehicle and drug treatment in control and stressed animals were calculated. Non-responders were identified by comparison of means of sucrose consumption at the end of drug treatment and analysis of variance with treatment (vehicle/drug) as the between-subject factor. Mean cumulative sucrose consumption in the animals responding to drug treatment was subsequently used an indicator for onset of drug action.

It was found that chronic mild stress induced a decrease in the consumption of 1% sucrose solution. In the final baseline test animals drank approximately 15-16 g of sucrose solution. Following initial two weeks of stress, the intakes remained at the same level in control rats but fell to approximately 8 g in stressed animals. In vehicle-treated animals the difference between control and stressed animals persisted at the same level for the remainder of the 5-weeks treatment period. Treatment with citalopram (2.5 or 5.0 mg/kg), or citalopram (2.5 or 5.0 mg/kg) combined with pyridoxine (10 mg/kg) showed no significant effect on sucrose intake in control animals, but increased the sucrose consumption, with different patterns of efficacy, back to normal levels in stressed animals.

As shown in FIG. 1, citalopram treatment combined with 10 mg/kg pyridoxine yielded the highest responder rates in stressed animals, with 6 out of 8 rats (75%) exhibiting increased sucrose intake in citalopram (2.5 mg/kg)/pyridoxine (10 mg/kg) and citalopram (5.0 mg/kg)/pyridoxine (10 mg/kg) groups. In comparison, only 4 out of 8 rats (50%) responded similarly to citalopram at 2.5 mg and 5 out of 8 rats (62.5%) at 5.0 mg when citalopram was administered alone.

[image]

Interestingly, when the stressed animals that responded to drug treatment were further analysed for sucrose consumption over the entire treatment period, citalopram (2.5 mg/kg)/pyridoxine (10 mg/kg) and citalopram (5.0 mg/kg)/pyridoxine groups showed faster increase in sucrose intake and regain of normal sucrose consumption rates (FIG. 2). Starting already at week 2 of drug treatment, values for mean cumulative sucrose intake are higher in animals treated with citalopram (2.5 mg/kg)/pyridoxine (10 mg/kg) and citalopram (5.0 mg/kg)/pyridoxine groups than in stressed animals treated with citalopram 2.5 mg/kg or 5 mg/kg. Following the consecutive treatment weeks, this difference is maintained and further expanded in favour of the citalopram (2.5 mg/kg)/pyridoxine (10 mg/kg) and citalopram (5.0 mg/kg)/pyridoxine groups, demonstrating that by combined citalopram and pyridoxine treatment the weekly sucrose intake level of unstressed controls is reached faster.

[image]

In conclusion, these data show that with combined treatment of SRI and pyridoxine the responder rate and the onset of action are both improved in comparison to single SRI treatment in an animal model of depression.

### Example II

### Onset of Action

Patient Selection

Patients included in this study have been diagnosed as having Major Depressive Disorder using criteria as set forth in the DSM-IV. Two psychiatrists have confirmed this diagnosis independently before inclusion. All patients are between the ages of 18 and 75 years, are not vitamin B6 deficient and have no major medical problems nor are they using any medication to treat their depression.

The severity of the depression of the patients is assessed at baseline using the Hamilton Rating Scale for Depression (HAM-D 17 items) (for review: Hamilton M. (1960) “A rating scale for depression”. J. Neurol. Neurosurg. Psychiatry 23, 56; Hamilton M. (1967) “Development of a rating scale for primary depressive illness”. Br J Soc Clin Psychol 6, 278-296). Only patients with a score greater than 21 on the HAM-D 17 continue in the study (a HAM-D 17 score of 18-25 indicates moderate to severe depression). Based on these criteria, twenty (20) patients with a depressive episode are selected for this study.

Dose Regimen

The patients are randomised to treatment groups A or B. Each group receives a different daily dose regimen, i.e.:

A: 20 mg fluoxetine+500 mg vitamin B6; and

B: 20 mg fluoxetine+placebo (appearance of the placebo is identical to the tablet containing vitamin B6)

Scoring

During the study period of 10 weeks, patients are assessed using the HAM-D at weekly intervals by experienced raters.

Results

On average, the HAM-D scores of patients in treatment group A are found to decline faster than in treatment group B. This indicates that the regimen of group A has a positive effect on the onset of action of fluoxetine.

### Example III

### Non-Responders

Patient Selection

Patients included in this study were selected on the basis of the same criteria as described in example I.

After 6 weeks of adequate SSRI therapy (i.e., fluoxetine 20 mg daily), those patients who showed insufficient improvement on the HAM-D 17, are considered to be ‘non-responders’. Twenty (20) ‘non responders’ are selected for inclusion in the present study.

Dose Regimen

The patients are randomised to treatment groups A or B. Each group receives a different daily dose regimen, i.e.:

A: 20 mg fluoxetine+500 mg vitamin B6; and

B: 20 mg fluoxetine+placebo

Scoring

During the study period of 8 weeks, patients are assessed using the HAM-D 17.

Results

The number of patients showing a decrease of baseline HAM-D scores of 50% or greater in group A is higher than that in group B. This indicates that the regimen of group A enhances the response to fluoxetine treatment in patients considered to be non-responders.

### Example IV

### Onset of Action (Low Dose of SSRI)

Dose Regimen

The patients are randomised to treatment groups A or B. Each group receives a different daily dose regimen, i.e.:

A: 10 mg fluoxetine+500 mg vitamin B6; and

B: 10 mg fluoxetine+placebo

Scoring

During the study period of 10 weeks, patients are assessed using the HAM-D 17.

Results

The patients in group A are found to show a faster decline in the HAM-D scores than those in treatment group B. This indicates that the regimen of group A has a positive effect on the onset of action of low dosed fluoxetine.

1-17. (canceled)

18. A method of improving the responder rate to or advancing the onset of action of serotonin re-uptake inhibitor (SRI) in the treatment of depressive disorders, anxiety disorders, post-traumatic stress syndrome or premenstrual syndrome, said method comprising combined administration of SRI in a daily amount of at least 0.4 mg, and vitamin B6 component in a daily amount of between 0.01 and 10 mmoles, and wherein the method does not include the application to the brain of an AC pulsed magnetic field of at least 7.5 picotesia flux density for at least 15 minutes.

19. The method according to claim 18, for use in the treatment of depression or anxiety disorders.

20. The method according to claim 18, wherein the vitamin B6 component is co-administered in a daily amount of between 0.01 and 10 mmoles to advance the onset of action of the treatment with SRI.

21. The method according to claim 18, wherein the method comprises at least once daily administration of the SRI and the vitamin B6 component.

22. The method according to claim 18, wherein the method comprises administration of SRI in a daily amount of between 0.01 and 1 mg per kg bodyweight and vitamin B6 component in a daily amount of between 0.001 and 0.2 mmoles per kg bodyweight.

23. The method according to claim 18, wherein the vitamin B6 analogue is selected from the group consisting of pyridoxal, pyridoxamine, acetals of pyridoxal, condensation products arising from the reaction of the aldehyde group of pyridoxal with an amine, and addition salts of any of the foregoing members of the group with pharmaceutically acceptable salts.

24. The method according to claim 18, wherein the dosage ratio of vitamin B6 component to serotonin re-uptake inhibitor is in the range of 0.01 to 1 mmole/mg.

25. The method according to claim 18, wherein the serotonin re-uptake inhibitor is selected from the group consisting of citalopram, escitalopram, fluoxetine, norfluoxetine, fluvoxamine, paroxetine, sertraline, venlafaxine, zimelidine, femoxetine, trazodone, nefazodone, duloxetine, pharmaceutically acceptable salts of these inhibitors and mixtures thereof.

26. The method according to claim 18, wherein the formulation comprises at least 300 mg of a serotonin precursor selected from the group consisting of L-tryptophan, 5-hydroxytryptophan, precursors thereof and mixtures thereof.

27. The method according to claim 18, wherein the formulation comprises at least 0.05 mmoles of a salicylate.

28. The method according to claim 18, wherein the formulation does not contain a narcotic selected from the group consisting of codeine, oxycodone, propoxyphene, pentazocine, morphine, merperidine, levorphanol, methadone and mixtures thereof, in an amount effective to produce analgesia.

29. The method according to claim 18, wherein the method comprises the at least once daily oral administration of a slow release formulation containing the vitamin B6 component.

30. A pharmaceutical formulation containing at least 0.4 mg of serotonin re-uptake inhibitor, at least 0.01 mmole of vitamin B6 component and a pharmaceutically acceptable carrier, said vitamin B6 component being contained in a dosage unit that provides a sustained release of the vitamin B6 component.

31. The pharmaceutical formulation according to claim 30 for oral, rectal, buccal or transdermal administration, comprising a solid dosage form that contains as active agents at least 0.4 mg of serotonin re-uptake inhibitor and at least 0.01 mmole of vitamin B6 component as well as a pharmaceutically acceptable carrier, said dosage form providing a sustained release of the vitamin B6 component.

32. The pharmaceutical formulation according to claim 31, wherein the solid dosage form provides a sustained release profile wherein less than 50 wt. % of the vitamin B6 component is released from the dosage form within the first 4 hours after administration and more than 80 wt. % of the vitamin B6 component is released from the dosage form within 24 hours.

33. The pharmaceutical formulation according to claim 31, wherein the solid dosage form releases the vitamin B6 component at a rate of at least 0.001 mmole/hour during the first 4 hours after administration.

34. The pharmaceutical formulation according to claim 30, wherein the vitamin B6 component and the serotonin re-uptake inhibitor are present in a ratio of between 0.01 and 1 mmole/mg.
